(19) United States
(12) Patent Application Publication
(10) Pub. No.: US 2006/0173304 A1
(43) Pub. Date: Aug. 3, 2006

(54) Volumetric ultrasound scanning of smaller-sized breast

(76) Inventor: Shih-Ping Wang, Los Altos, CA (US)

Correspondence Address: COOPER & DUNHAM, LLP, 1185 AVENUE OF THE AMERICAS, NEW YORK, NY, 10036, US

(21) Appl. No.: 10/532,568

(22) PCT Filed: Nov. 25, 2003
(86) PCT No.: PCT/US03/37864

§ 371 Date: Oct. 31, 2005

Related U.S. Application Data
(60) Provisional application No. 60/429,728, filed on Nov. 27, 2002.

(30) Foreign Application Priority Data
Nov. 27, 2002 (US) 10/305,936

(51) Int. Cl. A61B 8/00

(52) U.S. Cl. 600/437

(57) ABSTRACT

An apparatus and related methods are described for obtaining volumetric ultrasound scans of a breast of a supine patient in which a fluid reservoir including a bottom flexible membrane contacts an upward-facing surface of the breast. The fluid reservoir is filled with water or other suitable acoustically conductive fluid until the bottom membrane covering the breast is submerged. A transducer surface of an ultrasound probe is submerged in the fluid and moved over and/or around the breast area to obtain the ultrasound scans. Patient comfort is promoted, the patient being able to relax in a supine position during the procedure with a substantially uncompressed breast. Imaging near the chest wall is enhanced, especially for patients having smaller-sized breasts. Also described are preferred embodiments using multiple transducer surfaces in which shadowing effects are reduced.

### CROSS-REFERENCE TO RELATED APPLICATIONS

This application is a continuation-in-part of U.S. Ser. No. 10/305/936 filed Nov. 27, 2002, which is a continuation-in-part of U.S. Ser. No. 10/160,836 filed May 31, 2002, which is a continuation-in-part of International Application Ser. No. PCT/US01/43237, filed Nov. 19, 2001, which claims the benefit of U.S. Provisional Application No. 60/252,946 filed Nov. 24, 2000, each of the above being incorporated by reference herein. This application also claims the benefit of U.S. Provisional Application No. 60/429,728 filed Nov. 27, 2002, which is incorporated by reference herein.

### FIELD

The present specification relates to medical ultrasound imaging. More particularly, the present specification relates to an apparatus and method for reliably and comfortably obtaining volumetric ultrasonic scans of smaller-sized breasts, although it is to be appreciated that the preferred embodiments described herein can accommodate medium and large-sized breasts as well.

### BACKGROUND

For breast cancer screening purposes or other useful medical purposes it is often desirable to create a three-dimensional or volumetric digital representation of the sonographic properties of a breast from ultrasonic scans thereof. It is desirable for the ultrasonic scans to yield raw data from which an accurate volumetric representation can be computed, the volumetric representation comprising voxels containing acoustic impedance measurements of corresponding volume elements in the breast.

Challenges faced in designing a successful device for obtaining accurate volumetric ultrasound scans include: (i) a requirement to maintain direct acoustic coupling between an ultrasound transducer array and the breast surface that is free of air gaps; (ii) a requirement for the breast to remain motionless during the scanning process relative to a coordinate system of the volumetric representation; (iii) dealing with problems caused by shadowing effects behind acoustically dense tissues (e.g., nipple shadow); (iv) keeping the process as painless as possible for the patient; (v) keeping equipment costs under control; (vi) accommodating differently-size breasts, including smaller-sized breasts; and (vii) imaging the breast tissue at all locations where suspicious lesions might be located, including regions near the chest wall.

Proposals for acquiring volumetric ultrasound scans include U.S. Pat. No. 5,851,180 (Crosby et. al.), a drawing from which is reproduced herein at FIG. 1. The breast 100 of an upright woman (i.e., standing or sitting) is compressed between two rigid plates 41 and 50 while an ultrasound probe 53 is swept across the bottom of the breast. For clarity of illustration herein, each drawing is accompanied by x-y-z reference axes having a common convention in which (i) the z-axis runs from the anterior to the posterior of the patient, +z direction extending outward from the chest wall, (ii) the y-axis runs in the head-to-toe direction of the patient, with the +y direction extending upward toward the head, and (iii) the x-axis runs in the left-to-right direction of the patient. Accordingly, the x-y plane is parallel to the coronal plane, the x-z plane is parallel to the axial plane, and the y-z plane is parallel to the sagittal plane.

One of the difficulties with the proposal of FIG. 1 is a limited ability to image near the chest wall. Another difficulty with the proposal of FIG. 1 is patient discomfort in having the breast compressed between the rigid plates. The proposal of FIG. 1 is also difficult to apply to small-breasted women whose breasts cannot extend very far out onto the rigid plates. Another disadvantage of the proposal of FIG. 1 is problems with shadowing effects, since the interrogating acoustic pulses only come from a single direction.

Another proposal for acquiring volumetric ultrasound scans is discussed in WO 02/089672 (Kantorovich et. al.), a drawing from which is reproduced herein at FIG. 2. In the proposal of WO 02/089672 the patient 12 lies prone on a table with breast 14 suspended through a hole and into a cavity filled with an acoustically conductive liquid. Ultrasound transducer arrays are connected in various ways to a rotating structure that rotates around the z-axis, the interrogating pulses being sent horizontally into the breast from different directions around the z-axis. Although shadowing effects are avoided in the proposal of FIG. 2, there is still a disadvantage for small-breasted women whose breasts can not hang down far enough into the cavity to get imaged by the transducer arrays. Additionally, there is a limited ability to image the breast up near the chest wall.

Accordingly, it would be desirable to provide an ultrasound scanning apparatus that achieves accurate ultrasound scans of a breast volume even for small-breasted women.

It would be further desirable to provide an ultrasound scanning apparatus that provides a comfortable experience for the patient.

It would be still further desirable to provide such an ultrasound scanning apparatus that can image close to the chest wall, that is cost-efficient to fabricate, and that does not suffer from shadowing effects such as nipple shadow effects.

### SUMMARY

According to one preferred embodiment, a breast ultrasound adapter is provided for facilitating the reliable acquisition of breast ultrasound scans. The breast ultrasound adapter comprises an open fluid reservoir defined by side walls and a bottom membrane for contacting a breast surface. The breast ultrasound adapter is designed for placement on the breast of a supine patient such that it can be filled with water and/or a water-containing polymeric gel or other suitable fluid until the bottom membrane covering the breast is submerged. A transducer surface of an ultrasound probe submerged in the fluid is swept in a lateral direction across the breast and remains submerged during the lateral sweep. In one preferred embodiment, the transducer surface is maintained in a plane parallel to the fluid surface and does not contact the bottom membrane during the lateral sweep. Advantageously, the breast is maintained in a substantially consistent position and in a consistent state of compression during the scanning process, thereby facilitating consistency among the ultrasound slices that can be used to form a volumetric sonographic representation of the breast.

Water, water-containing polymeric gel, and/or oil are preferably used to improve the contact between the bottom membrane and the breast. Optionally, acoustic transducers such as audio speakers are fixed to the breast ultrasound adapter for use in vibrational resonance applications. In one preferred embodiment, the ultrasound probe is swept by hand across the top surface while position sensors detect its position. In another preferred embodiment, a mechanically driven probe is coupled to the top of the breast ultrasound adapter and the probe position is sensed by mechanical transducers to form an at least semi-automated breast ultrasound scanning device.

According to another preferred embodiment, multiple linear transducer arrays are submerged that define a common plane, at least one of the linear transducer arrays being oriented differently within that plane than at least one other of the linear transducer arrays by a first minimum angular difference such as 30 degrees. At least one of the linear transducer arrays is oriented a second minimum angle, such as 45 degrees, from an anterior-posterior axis of the patient. The transducer arrays are mechanically translated and/or rotated within the acoustically conductive fluid around the breast by an amount sufficient to allow each location in the breast to be imaged from at least two different directions. Two-dimensional ultrasound image slices generated in the common plane are compounded with each other in a manner that obviates shadowing effects. The resultant compounded two-dimensional images are then stacked so as to form a volumetric representation of the breast.

According to another preferred embodiment, at least two of the linear transducer arrays are non-planar with respect to each other. In this case, the resultant ultrasound slices are compounded directly in three-dimensional space during the formation of the volumetric ultrasound representation.

An ultrasound scanning apparatus according to the preferred embodiments is comfortable for the patient, who can relax in a supine position during the procedure with an uncompressed breast. Imaging near the chest wall is enhanced by virtue of the geometry of the transducer array loci during the scanning procedure. Imaging of smaller-sized breasts is accommodated, and indeed the results are superior for smaller-sized breasts with respect to imaging near the chest wall. In the preferred embodiments in which multiple transducers are used, shadowing effects are obviated. Moreover, because the imaging can be achieved with conventional 1-D ultrasound transducer arrays, device costs are substantially reduced as compared to devices that depend on more exotic 2-D transducer arrays or “1.5-D” transducer arrays that are substantially more expensive and more difficult to produce. It is to be appreciated, however, that such 2-D or 1.5-D arrays could indeed be used with the preferred embodiments if so desired and if their cost factors are not problematic.

### BRIEF DESCRIPTION OF THE DRAWINGS

FIG. 1 illustrates a prior art breast ultrasound scanning apparatus;

FIG. 2 illustrates a breast ultrasound scanning apparatus;

FIG. 3 illustrates a breast ultrasound adapter according to a preferred embodiment;

FIG. 4 illustrates a breast ultrasound scanning device according to a preferred embodiment;

FIG. 5 illustrates an ultrasound scanning apparatus according to a preferred embodiment;

FIG. 6 illustrates an ultrasound scanning apparatus according to a preferred embodiment;

FIG. 7 illustrates an ultrasound scanning apparatus according to a preferred embodiment;

FIG. 8 illustrates an ultrasound scanning apparatus according to a preferred embodiment; and

FIG. 9 illustrates an ultrasound scanning apparatus according to a preferred embodiment.

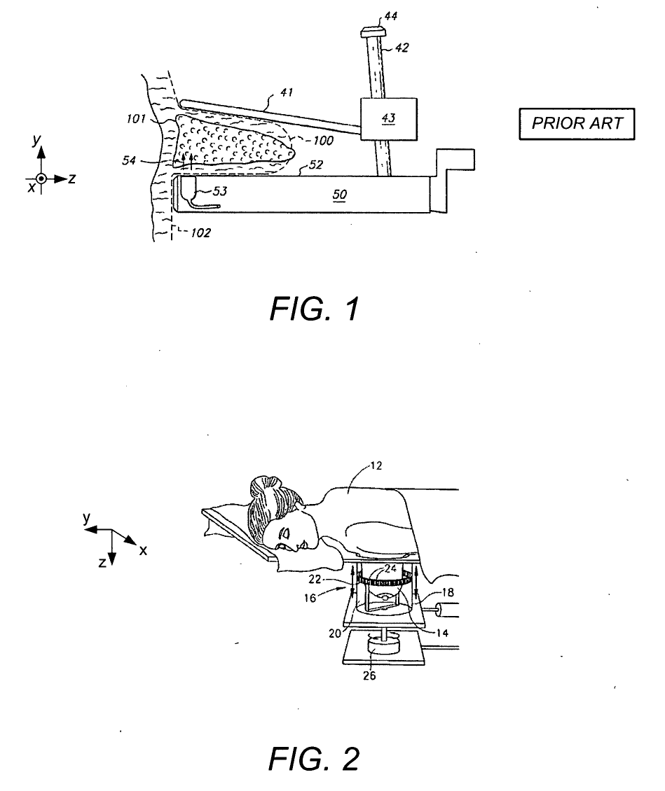

### DETAILED DESCRIPTION

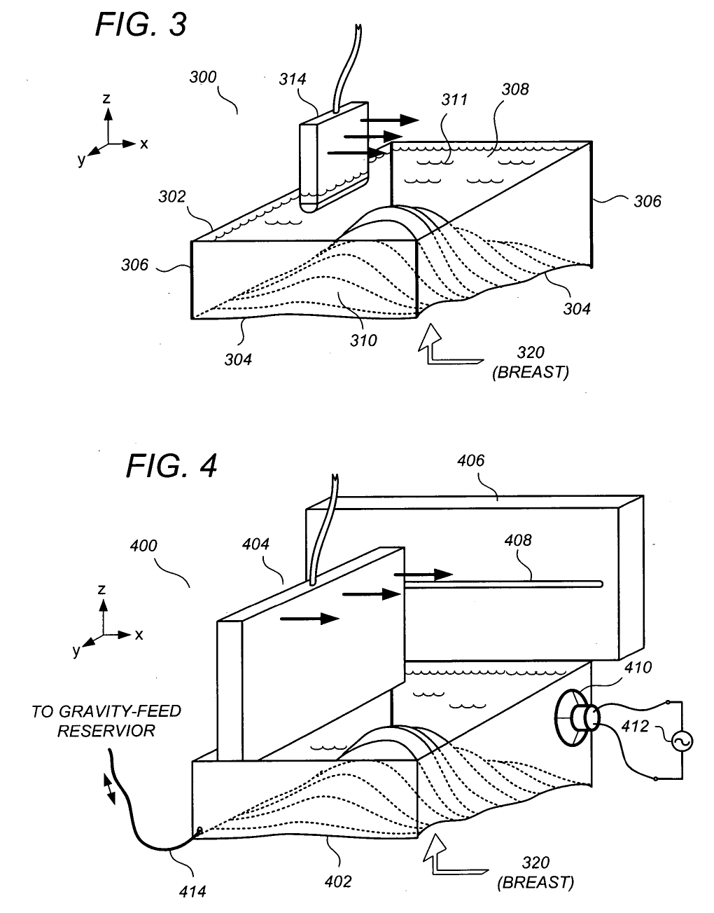

FIG. 3 illustrates a breast ultrasound adapter 300 for facilitating breast ultrasound scans in accordance with a preferred embodiment. As known in the art, it is required that a close acoustical coupling be maintained between the breast skin surface and the ultrasound probe at all times during the scan. In particular, there should be no vacuum or air pockets anywhere along the path of the interrogating ultrasound pulses. This is usually achieved by keeping the breast surface wet with a water-containing polymeric fluid or gel at all points of contact with the ultrasound probe. Alternatively, gel bags have been placed between the ultrasound probe and skin surface to maintain an acoustic coupling. With both of these methods, however, the breast itself is necessarily shifted around and compressed by differing amounts at differing places as the ultrasound probe is moved.

Breast ultrasound adapter 300 comprises an upper frame 302, side walls 306, and a bottom membrane 310 forming a fluid reservoir volume 308 for holding a water-containing polymeric fluid or gel. As shown in FIG. 3, breast ultrasound adapter 300 has been placed over a breast 320 and filled with fluid 311, and the bottom membrane 310 has conformally adapted to the shape of the breast 320. The fluid level should rise above the highest point of the breast 320. The bottom of an ultrasound probe 314 is submerged in the fluid 311 and is swept in a lateral direction across the breast as indicated in FIG. 3, the bottom of the ultrasound probe 314 remaining submerged during the lateral sweep. Preferably, the bottom of ultrasound probe 314 is maintained in a plane parallel to the fluid surface and does not contact the bottom membrane 310 during the lateral sweep. Position sensors (not shown) are used to track the position of the ultrasound probe 314 as it is swept, usually by hand, across the fluid surface. Preferably, water-containing polymeric gel, water, and/or oil are used to improve the contact between the bottom membrane 310 and the breast 320. Advantageously, the breast is maintained in a substantially consistent position and in a consistent state of compression during the scanning process, thereby facilitating consistency among the ultrasound slices forming the three-dimensional image volume.

The breast ultrasound adapter 300 may comprise any of a variety of material configurations that facilitate the presence of an open fluid reservoir above the breast of a supine patient, the fluid reservoir having a lower surface that conforms to the breast shape such that an ultrasound probe is acoustically coupled to the breast skin surface when immersed in the fluid. By way of example, upper frame 302 is preferably a rigid or semi-rigid compression-molded silicone rubber material, or an equivalent material, such that the breast ultrasound adapter 300 can be supported and moved by manipulating the upper frame 302 even when full of fluid. Side walls 306 may also be rigid or semi-rigid. In the embodiment of FIG. 3, side walls 306 are semi-rigid and sufficiently flexible such that they are conformal with the patient's skin surface along points of intersection 304 therewith. However, in alternative preferred embodiments, the side walls 306 do not contact the skin surface directly and therefore may be rigid. In this case, the bottom membrane protrudes downward from the side walls and hangs down like a plastic bag over the breast when filled with fluid. In still other preferred embodiments, the side walls 306 are not present at all, and the bottom membrane hangs down directly from the upper frame 302 over the breast when filled with fluid. In this preferred embodiments in which the side walls 306 do not contact the skin surface directly or are not present, the breast ultrasound adapter 300 is supported by the upper frame 302 and laid gently over the breast, which does not support its entire weight. The bottom membrane 310 preferably comprises a flexible, watertight, conforming material. An at least partially distensible characteristic assists in ensuring bubble-free contact with the breast skin. Suitable materials include protective latex, synthetic elastomers, cellophane, or other protective sheath-type materials described in U.S. Pat. No. 6,039,694, which is incorporated by reference herein. The respective pieces are fastened together by a suitable adhesive such as silicone RTV.

FIG. 4 illustrates a breast ultrasound adapter 402 having additional features in accordance with a preferred embodiment that, in conjunction with a mechanical probe translation assembly, forms a semi-automatic breast ultrasound scanning device 400. Breast ultrasound adapter 402 comprises an integrated audio speaker 410 coupled to an electrical source 412 for use in vibrational resonance applications. The apparatus of FIG. 4 also comprises a conceptual diagram of an ultrasound probe 404 as it is guided by a mechanical translator 406, for example by means of a slot 408. Advantageously, the probe position may be sensed by mechanical transducers instead of position sensors as required in the embodiment of FIG. 3, supra. The mechanical translator may be affixed to the frame of the breast ultrasound adapter 402, or may alternatively be externally supported.

Finally, breast ultrasound adapter 402 further comprises a fluid transport opening and fluid conduit 414 for coupling to a gravity-feed reservoir (not shown) that is functionally similar to an intravenous (IV) fluid container. In accordance with a preferred embodiment, the gravity-feed reservoir is raised, either by hand or by an automated mechanical assembly, to introduce fluid into the breast ultrasound adapter 402 after it has been placed on a patient's breast. Following the scan procedure, the gravity-feed reservoir is lowered to cause fluid to drain out, thereby emptying the breast ultrasound adapter 402. The vertical position of the gravity-feed reservoir may also be adjusted used to regulate the level of the fluid during the scan.

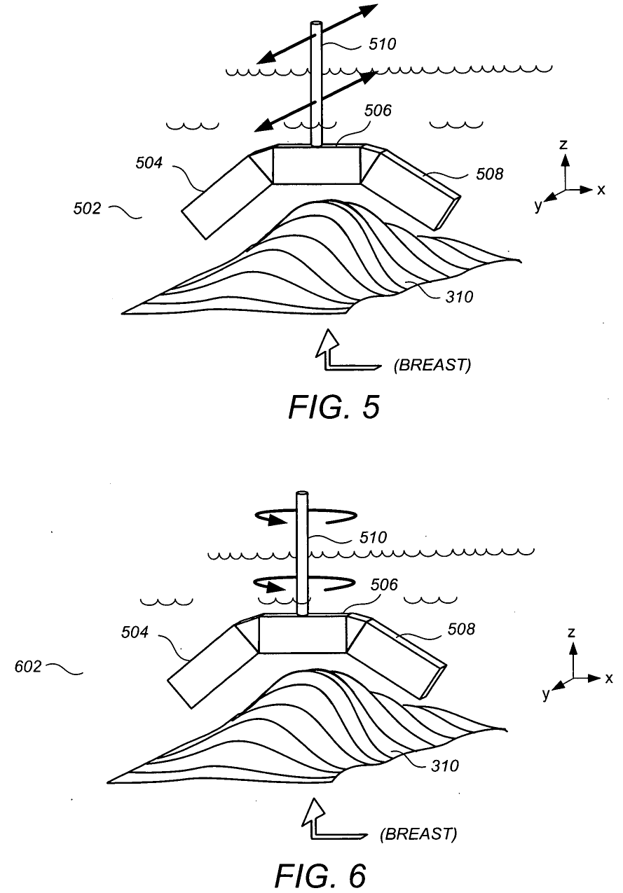

FIG. 5 illustrates an ultrasound scanning apparatus 502 according to a preferred embodiment. The ultrasound scanning apparatus 502 generally comprises elements similar to those of FIGS. 3 and/or 4 above, except that a different mechanical configuration using multiple linear transducer arrays is provided that can be used to obviate shadowing effects. Ultrasound scanning apparatus 502 comprises three linear ultrasound transducer arrays 504, 506, and 508 that are rigidly connected and arranged as shown in FIG. 5. By virtue of support element 510 rigidly connected to the center transducer array 506 and connected to external actuation devices (not shown), the transducer assembly is supported and mechanically actuated. It is to be appreciated that the fluid reservoir assembly and the acoustically conductive liquid submersing the transducer assembly is also provided, but the reservoir frame is omitted in FIGS. 5-9 for clarity of presentation. Also, the form factors of the transducer arrays are simplified, and miscellaneous items such as probe wires are omitted, for clarity of presentation.

In operation, the scanning apparatus 502 is linearly translated in the y-direction by the external actuation devices such that the position of the assembly for each scan is recorded and maintained for use in forming the volumetric representation. According to a preferred embodiment, since there are three linear ultrasound arrays, there are three sets of ultrasound slices obtained which are then spatially compounded to achieve a single ultrasound slice. The ultrasound slices are then stacked so as to form a volumetric representation of the breast. Known methods may be used to obtain the compounded slices from the raw, uncompounded ultrasound slices. Notably, in view of the orientation of the transducer arrays relative to the small-breasted woman, it is readily seen that imaging back to the chest wall is facilitated.

During the scanning process, the transducers 504, 506, and 508 should be operated at different time intervals to prevent mutual acoustic interference. For example, the acoustic pulses from the respective transducers can be interleaved in time in an a-b-c-a-b-c sequence during a single probe sweep. Alternatively, the transducer arrays can be separately actuated during separate sweeps. Advantageously, since the breast is not being compressed and since the woman is lying comfortably on her back, there is no particular urgency to finish the scanning process quickly, provided that the woman remains substantially motionless.

According to one preferred embodiment, the effect of the compounding process itself obviates most nipple shadowing effects and other shadowing effects, increasing the overall accuracy of the images. According to another preferred embodiment, for each voxel, the maximum reading obtained among the three transducer array readings (corrected for distance factors) is used. According to yet another preferred embodiment, each separate ultrasound slice can be processed to detect shadowing effects by first low-pass filtering that ultrasound slice and then searching for shadowing effects in the known direction facing away from that particular transducer array. Once the shadowed areas are known for a given transducer array's contribution, the volumetric construction algorithms can ignore that transducer array's contribution when computing the compounded ultrasound slice/volumetric representation. Other methods for compounding the raw ultrasound readings from the three separate transducer arrays, such as those based on statistical methods, are also within the scope of the preferred embodiments.

FIG. 6 illustrates an ultrasound scanning apparatus 602 according to another preferred embodiment, similar to the embodiment of FIG. 5 except that the assembly of transducer arrays is rotated around an axis parallel to the z-axis instead of, or in addition to, being horizontally translated in the x-y plane. Notably, it is not required that the axis of the support element 510 be placed directly over the nipple of the breast. As long as the position of the scanning apparatus is known for each set of raw ultrasound slices, an accurate volumetric representation of the breast can be achieved.

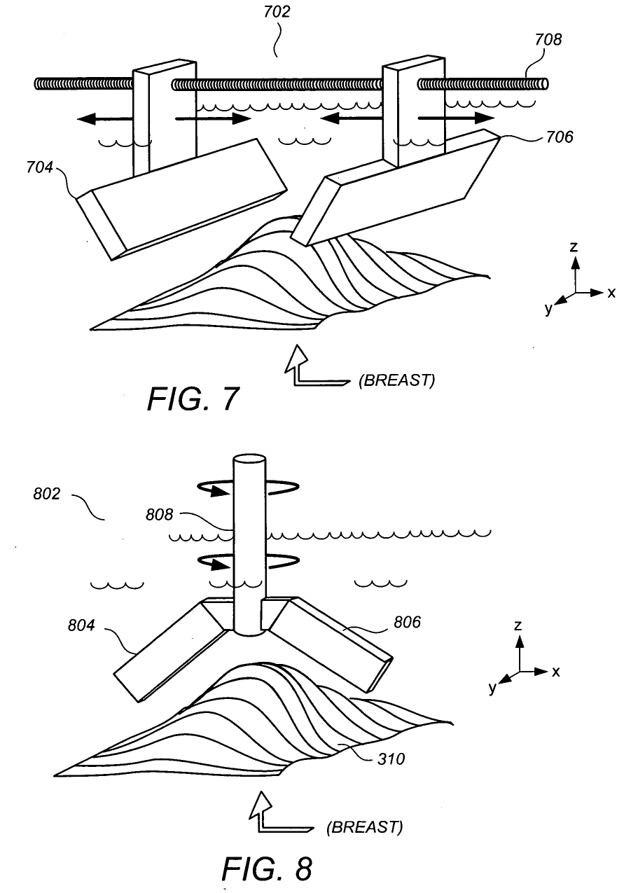

FIG. 7 illustrates an ultrasound scanning apparatus 702 according to another preferred embodiment in which longer transducer arrays 704 and 706 are used which may be, for example, 15 cm-long 256-element arrays. In this embodiment the transducers arrays 704 are tilted inward, for example by about 15 degrees, away from y-z plane. The assembly is translated in the x-direction using any of a variety of known translation mechanisms such as a worm gear 708.

In an alternative preferred embodiment, the transducers 704 and 706 can be held stationary by the worm gear 708, while the angle of the transducers 704 and 706 is varied from approximately 0 degrees to approximately 90 degrees with respect to the y-z plane during the ultrasound scans. In such preferred embodiment, it would look like the transducers 704 and 706 are “fanning” or “waving at” the breast volume.

FIG. 8 illustrates an ultrasound scanning apparatus 802 to according to another preferred embodiment. Scanning apparatus 802 is similar to the embodiment of FIG. 6 except that two longer transducer arrays 804 and 806 are used instead of the three shorter transducer arrays.

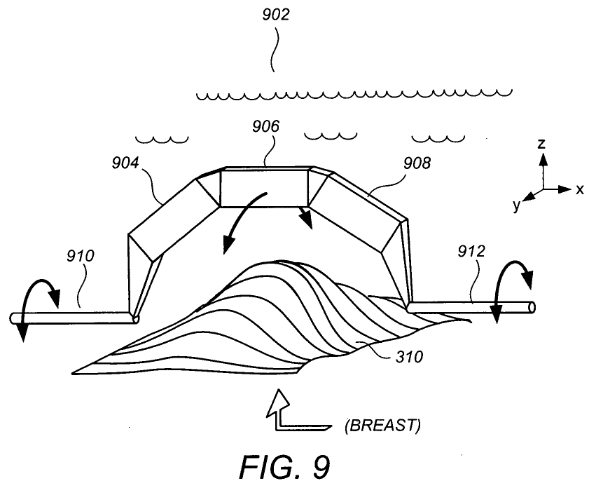

FIG. 9 illustrates an ultrasound scanning apparatus 902 according to another preferred embodiment. Three linear transducer arrays 904, 906, and 908 are integrated as shown into a rigid mechanical assembly fixedly attached to support members 910 and 912. The support members 910 and 912 lie along a common axis and are coupled to bearings and mechanical actuators (not shown) capable of at least partial rotation around the x-axis. During the scanning process, the support members 910 and 912 are rotated back and forth so as to subtend a 180-degree arc from one side of the breast to the other. The trajectory loci of the transducer arrays 904, 906, and 908 collectively form a dome-like shape over the breast.

Whereas many alterations and modifications of the present invention will no doubt become apparent to a person skilled in the art after having read the foregoing description, it is to be understood that the particular embodiments shown and described by way of illustration are in no way intended to be considered limiting. By way of example, while the breast ultrasound adapter described supra covers a single breast, in other preferred embodiments it is configured to cover both breasts simultaneously, thereby further expediting the ultrasound scanning process. By way of further example, the above preferred embodiments can be adapted for a prone position instead of a supine position of the patient, while still being advantageous over the proposal FIG. 2 at least because imaging near the chest wall is improved.

By way of still further example, while a preferred embodiment is described above in which coplanar linear transducer arrays are oriented in different directions within the common plane for reducing shadowing effects, beamsteering may be used in another preferred embodiment to achieve similar results. Thus, for example, the linear transducer arrays 804 and 806 in FIG. 8 can be collinear so as to form a “T” instead of a “Y” with the support member/shaft 808, and beamsteering at inward-facing angles can be used to generate the raw ultrasound scans that are compounded to reduce shadowing effects. Therefore, reference to the details of the preferred embodiments are not intended to limit their scope, which is limited only by the scope of the claims set forth below.

What is claimed is:

1. An apparatus for facilitating ultrasound scans of a breast of a supine patient, comprising a reservoir having a conformable, acoustically conductive membrane including a first area contacting an upward-facing surface of the breast, the reservoir maintaining an acoustically conductive fluid at a surface level sufficient to submerge said first area such that a transducer surface, when submerged into the fluid, comes into substantial acoustic communication with the upward-facing surface of the breast.

2. The apparatus of claim 1, the transducer being manually submerged and manipulated to acquire the ultrasound scans, said apparatus further comprising a position sensing system obtaining transducer location information corresponding to the ultrasound scans, whereby a volumetric representation of the breast can be computed using said ultrasound scans and said corresponding location information.

3. The apparatus of claim 2, wherein said ultrasound scans are planar scans, and wherein said transducer comprises a linear array transducer.

4. The apparatus of claim 3, further comprising a vibrational resonance audio source acoustically integrated into said reservoir.

5. The apparatus of claim 1, further comprising a mechanical manipulator moving the transducer beneath said surface level to acquire the ultrasound scans, whereby a volumetric representation of the breast can be computed using said ultrasound scans.

6. The apparatus of claim 5, wherein said ultrasound scans are planar scans, and wherein said transducer comprises a first linear array transducer.

7. The apparatus of claim 6, wherein said transducer further comprises a second linear array transducer having a common scanning plane with said first linear array transducer, said first and second linear array transducers having scanning directions within said common scanning plane that differ by a first nonzero angle, whereby outputs of said first and second linear array transducers can be compounded to reduce shadowing effects in the ultrasound scans.

8. The apparatus of claim 7, wherein said first nonzero angle is at least 30 degrees.

9. The apparatus of claim 7, wherein said mechanical manipulator translates said first and second linear array transducers in a direction substantially parallel to a coronal plane of the supine patient.

10. The apparatus of claim 7, wherein said mechanical manipulator rotates said first and second linear array transducers around an axis substantially perpendicular to a coronal plane of the supine patient.

11. The apparatus of claim 6, wherein said transducer further comprises a second linear array transducer having a scanning plane non-parallel to a scanning plane of said first linear array transducer, whereby first and second intermediate volumetric representations separately derived from outputs of said first and second array transducers, respectively, can be compounded to reduce shadowing effects in the ultrasound scans.

12. The apparatus of claim 11, wherein said mechanical manipulator translates said first and second linear array transducers in a direction substantially parallel to a coronal plane of the supine patient.

13. An apparatus for ultrasonically scanning a breast of a supine patient, comprising:
a probe assembly comprising first and second linear array transducers affixed thereto, said first and second linear array transducers having scanning directions within a common scanning plane that differ by a first nonzero angle;
a reservoir having a conformable, acoustically conductive membrane including a first area contacting an upward-facing surface of the breast, the reservoir maintaining an acoustically conductive fluid at a surface level sufficient to submerge said first area;
a mechanical manipulator positioning said probe assembly such that a scanning surface of each of said first and second linear array transducers is submerged in said fluid to achieve acoustic contact with the upward-facing surface of the breast, said mechanical manipulator moving said probe assembly during a scanning session such that said common scanning plane passes through substantially all of an interior volume of the breast.

14. The apparatus of claim 13, wherein said first nonzero angle is at least 30 degrees.

15. The apparatus of claim 13, wherein said mechanical manipulator translates said first and second linear array transducers in a direction substantially parallel to a coronal plane of the supine patient.

16. The apparatus of claim 13, wherein said mechanical manipulator rotates said first and second linear array transducers around an axis substantially perpendicular to a coronal plane of the supine patient.

17. An apparatus for ultrasonically scanning a breast of a supine patient, comprising:
a probe assembly comprising first and second linear array transducers having non-parallel scanning planes differing by a first nonzero angle;
a reservoir having a conformable, acoustically conductive membrane including a first area contacting an upward-facing surface of the breast, the reservoir maintaining an acoustically conductive fluid at a surface level sufficient to submerge said first area;
a mechanical manipulator positioning said probe assembly such that a scanning surface of each of said first and second linear array transducers is submerged in said fluid to achieve acoustic contact with the upward-facing surface of the breast, said mechanical manipulator actuating said probe assembly during a scanning session such that the scanning plane of each of said first and second linear array transducers passes through substantially all of an interior volume of the breast.

18. The apparatus of claim 17, wherein said first nonzero angle is at least 30 degrees.

19. The apparatus of claim 17, wherein said mechanical manipulator translates said first and second linear array transducers in a direction substantially parallel to a coronal plane of the supine patient.

20. The apparatus of claim 17, wherein said mechanical manipulator varies an elevation angle of the scanning plane for each of said first and second linear transducer arrays relative to a coronal plane of the supine patient.
